(19) United States
(12) Patent Application Publication
(10) Pub. No.: US 2025/0388942 A1
(43) Pub. Date: Dec. 25, 2025

(54) MICROBIAL BIOSYNTHESIS OF COMPOSITES

(71) Applicants: Weinan Xu, Hudson, OH (US); Shan Liu, Stow, OH (US); Hazel Barton, Akron, OH (US)

(72) Inventors: Weinan Xu, Hudson, OH (US); Shan Liu, Stow, OH (US); Hazel Barton, Akron, OH (US)

(21) Appl. No.: 19/244,400

(22) Filed: Jun. 20, 2025

Related U.S. Application Data
(60) Provisional application No. 63/661,762, filed on Jun. 19, 2024.

(51) Int. Cl. C12P 19/04; C01B 25/32; C12P 3/00

(52) U.S. Cl. CPC C12P 19/04; C01B 25/32; C12P 3/00

(57) ABSTRACT

A method of microbial biosynthesis of a composite includes subjecting a first culture within a bioreactor to incubation conditions, the first culture including a first bacteria that exhibit production of a structural polysaccharide, to thereby produce an organic network structure made of produced structural polysaccharide. A second culture is added to the bioreactor after the organic network structure is produced, the second culture including a second bacteria that exhibit mineralization of calcium carbonate via microbial-induced carbonate precipitation. The second bacteria produce calcium carbonate particles which precipitate on the organic network structure to produce a mineralized organic network structure as the composite. The calcium carbonate particles can be chemically converted to calcium hydroxyapatite particles.

### CROSS REFERENCE TO RELATED APPLICATIONS

This application claims the benefit of U.S. provisional patent application Ser. No. 63/661,762, filed Jun. 19, 2024, which is incorporated by reference herein.

### FIELD OF THE INVENTION

One or more embodiments of the invention are directed toward microbial biosynthesis of composites, such as bone-mimetic composites.

### BACKGROUND

Development of biomimetic composites seeks to create properties and functionalities that match or exceed natural materials. One source of inspiration for biomimetic composites is human bones. Human bones generally have a hierarchical structure including about 50% to 70% of inorganic components, which are primarily calcium hydroxyapatite (CaHA), and about 20 to 40% of organic constituents, which are primarily type I collagen.

For the primary mechanism of bone development, known as osteogenesis, collagen nanofibers and ground substances form bundles, and clusters of CaHA nanoparticles are incorporated in the collagen matrix. In the secondary mechanism of bone formation, the primary arrays are remodeled into a more optimal structure, such as concentric lamellae, that make up osteons. Finally, the osteons are either packed densely into compact bone or comprise a trabecular network of microporous bone, referred to as spongy or cancellous bone, respectively.

Bone tissue engineering, which may be referred to as producing bone-inspired composites, attempts to overcome the limited supply issue and size limitation of autografts or allografts. One conventional study demonstrated that bacterial cellulose (BC), bacterially-precipitated calcium carbonate (CaCO₃), and biosynthesized poly (g-glutamic acid) can be blended to form composites molded into different shapes. Another conventional effort assembled amyloid nanofibers and 2D CaHA nanoplatelets into nanocomposites by a vacuum filtration method. Aligned bacterial cellulose nanofibers have also been used as a template for chemical mineralization with hydroxyapatite. A further conventional effort included producing hydroxyapatite/polymethyl methacrylate (HA/PMMA) composites with nacre-like architectures produced with a bidirectional freeze-casting method and in situ polymerization.

There remains a need for improved microbial biosynthesis of composites, such as bone-mimetic composites.

### SUMMARY

An aspect of the present invention provides a method of microbial biosynthesis of a composite, where the method includes subjecting a first culture within a bioreactor to incubation conditions, the first culture including a first bacteria that exhibit production of a structural polysaccharide, to produce an organic network structure made of a produced structural polysaccharide; adding a second culture to the bioreactor after the organic network structure is produced, the second culture including a second bacteria that exhibit mineralization of calcium carbonate via microbial-induced carbonate precipitation; and allowing the second bacteria to produce calcium carbonate particles which precipitate on the organic network structure to produce a mineralized organic network structure as the composite. The calcium carbonate particles can be chemically converted to calcium hydroxyapatite particles.

### BRIEF DESCRIPTION OF THE DRAWINGS

Advantages of the present invention will become better understood with regard to the following description, appended claims, and accompanying drawings wherein:

FIG. 1 is a schematic of a method for microbial biosynthesis of a composite;

FIG. 2 is a scanning electron microscopy (SEM) image of a bacterial cellulose nanofiber network;

FIG. 3 is SEM images of bacterial cellulose and calcium carbonate composites, with the top image showing a composite prepared by a one-cycle biosynthesis and the bottom image showing a composite prepared by two-cycle biosynthesis;

FIG. 4 is SEM images of bacterial cellulose films made with different culturing times, increasing from 1 day in the top image, 3 days in the middle image, to 5 days in the bottom image;

FIG. 5 is SEM images of biosynthesized calcium carbonate particles in the top image, with the bottom image showing conversion to calcium hydroxyapatite particles; and

FIG. 6 is photographs of biosynthesized composites, with the top image showing a composite in a star cylinder shape and the bottom image showing a partial bone shape.

### DETAILED DESCRIPTION

Embodiments of the invention are based on microbial biosynthesis of composites. The microbial biosynthesis method creates a composite, which can be referred to as a 3D hierarchical composite, with controllable or programmable properties, such as size, shape, and internal structure. The programmable microbial biosynthesis method includes a stepwise culturing approach where a first bacteria produces an organic network structure made of a structural polysaccharide. The stepwise culturing technique then subjects the organic network structure to a second bacteria which exhibits mineralization of calcium carbonate (CaCO₃) via microbial-induced carbonate precipitation (MICP). This results in calcium carbonate particles precipitated on the organic network structure. The calcium carbonate particles can be chemically converted to calcium hydroxyapatite (CaHA) via a suitable reagent, such as where a bone-mimetic structure is desired. Advantageously, the microbial biosynthesis of organic-inorganic composites in embodiments of the present invention opens new avenues for the scalable creation of functional biomaterials in an efficient and sustainable way. The bioreactor utilized in the microbial biosynthesis can be made with an additive manufacturing technique, such as direct ink writing (DIW), which enables the fabrication of precise and customizable 3D hierarchical structure composites. This can be important for tissue engineering. Further, the method of producing a biomimetic composite advantageously allows for structural control with tunable porosity, density, and mechanical properties. In addition to bone-mimetic composites, the composites can therefore be useful in other applications, such as lightweight materials, green materials, thermal management, packaging, sensing, and other living material applications.

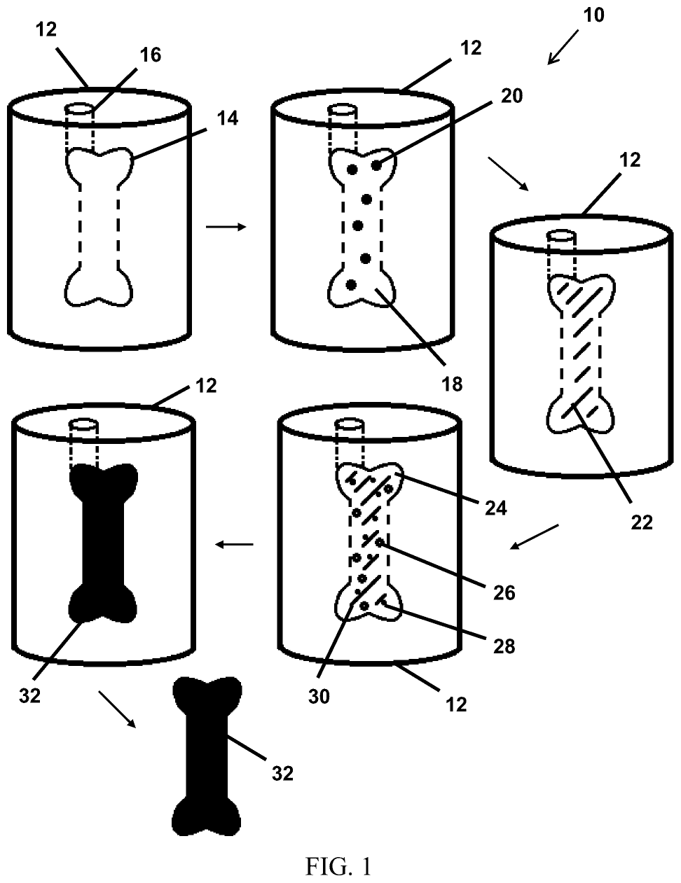

With particular reference to FIG. 1, one or more embodiments of the present invention provide a method 10 for microbial biosynthesis of a composite. Method 10 includes obtaining or making a vessel 12, which can be referred to as a bioreactor 12. Bioreactor 12 can be made by an additive manufacturing technique, such as direct ink writing. Where bioreactor 12 is made by additive manufacturing, bioreactor 12 should be made of a gas permeable material to allow a suitable amount of gas diffusion for the microbial biosynthesis.

Bioreactor 12 includes a hollow interior 14, and where it is desired to form a particular shape for the composite, hollow interior 14 can be shaped as the desired shape for the composite, as generally shown in FIG. 1. Hollow interior 14 may therefore also be referred to as shaped hollow interior 14 or inverse interior 14. In one or more embodiments, hollow interior 14 may be a larger overall shape, such as a cylinder, and the resulting overall shaped composite can be formed into the desired shape, such as by trimming.

Method 10 next includes adding, which can be via an inlet 16, a desired amount of a first culture 18, which may also be referred to as liquid media 18 or liquid culture 18. A variety of configurations for inlet 16 will be suitable, such as a cylinder as shown or a top of shaped hollow interior 14 serving as an inlet, or other configuration such as inlet piping. The amount of first culture 18 added can be an amount to partially fill or entirely fill shaped hollow interior 14.

First culture 18 includes a first bacteria 20 capable of producing an organic network structure 22, which can be made of a structural polysaccharide such as bacterial cellulose. That is, the first bacteria 20 is capable of producing bacterial cellulose or other suitable structural polysaccharide. The first bacteria 20 is subjected to incubation conditions for a desired time in order to form the organic network structure 22, which may be referred to as porous nanofiber network structure 22. Where the liquid culture 18 fills the shaped hollow interior 14, the organic network structure 22 will have a shape generally matching the shape of shaped hollow interior 14.

After the desired formation of organic network structure 22, a remaining portion of liquid culture 18 should be removed from bioreactor 12. The bioreactor 12 can then be suitably cleaned or rinsed, such as with sterile water. A desired amount of a second culture 24, which may also be referred to as liquid media 24 or liquid culture 24, is then added to hollow interior 14. The amount of second culture 24 added can be an amount to partially fill or entirely fill shaped hollow interior 14.

Second culture 24 includes a second bacteria 26 capable of mineralization of calcium carbonate (CaCO₃) 28, such as biologically induced mineralization (BIM) or biologically controlled mineralization (BCM), via microbial-induced carbonate precipitation. Techniques for microbial-induced carbonate precipitation include the calcium toxicity mechanism, urea hydrolysis, ammonification of amino acids, denitrification, and photosynthesis. Where the microbial-induced carbonate precipitation technique is the calcium toxicity mechanism, second culture 24 should include a calcium source in order to stress the second bacteria 26, which causes second bacteria 26 to precipitate calcium carbonate 28 to remove excess Ca²⁺ ions. In any of the microbial-induced carbonate precipitation techniques, calcium carbonate 28 should preferentially precipitate on the surface of the nanofibers of organic network structure 22. This results in the organic network structure 22, which may also be referred to as polymeric matrix 22, becoming mineralized with calcium carbonate particles 28 as a mineralized organic network structure 30.

After the desired formation of calcium carbonate particles 28 for mineralized organic network structure 30, a remaining portion of liquid culture 24 can be removed from bioreactor 12. An initial composite and/or bioreactor 12 can then be subjected to drying conditions in order to achieve a composite 32 which contains organic network structure 22 and inorganic calcium carbonate particles 28. The drying can include removing a majority, or substantially all, or all of the associated water. Drying the initial composite can be said to achieve composite 32. The drying conditions can include freeze drying. An exemplary freeze-drying technique includes subjecting to a temperature of about −50° C. for about 24 hours. Composite 32 is removed from bioreactor 12, such as by peeling away or cutting bioreactor 12 or other suitable technique, for use of composite 32. Since the material for bioreactor 12 can be relatively soft in some embodiments, exemplary cutting tools include a knife or scissors. The removal of composite 32 can be before, during, or after the drying conditions.

For certain end applications for composite 32, composite 32 can be modified either before or after removal from bioreactor 12. For example, where composite 32 is intended as a bone-mimetic structure, the calcium carbonate particles 28 can be chemically converted to inorganic calcium hydroxyapatite particles. Hydroxyapatite can provide additional hardness and strength relative to carbonate. Chemical conversion of calcium carbonate to calcium hydroxyapatite can be achieved via ammonium phosphate by the reaction: 10CaCO₃+6NH₄(H₂PO₄)+2H₂O→Ca₁₀(PO₄)6(OH)₂+3(NH₄)2CO₃+7H₂CO₃.

For certain end applications, such as tissue engineering applications, composite 32, which as mentioned above can also be a modified composite 32, can be treated for removal of bacterial endotoxin. Disintegration of first bacteria 20 and/or second bacteria 26 can include releasing undesirable components, such as exopolysaccharides (EPS), peptidoglycan, and lipopolysaccharide (LPS, for gram-negative bacteria). Method 10 can include treating or sanitizing composite 32 to remove these residual undesired components.

Further details of bioreactor 12 are now provided. The bioreactor 12 can be any suitable device or system capable of supporting a biologically active environment according to the disclosure herein. As further discussed herein, in one or more embodiments the bioreactor 12 is a polymer, such as an elastomer, made from an additive manufacturing process. In other embodiments, bioreactor 12 can be a metal vessel, such as made from stainless steel. The microbial biosynthesis of composite 32 requires sufficient access to gases, such as oxygen and carbon dioxide, and bioreactor 12 therefore should provide such access. In one or more embodiments, gases (i.e., oxygen and carbon dioxide) can be provided from atmospheric air. In these or other embodiments, gases (i.e., oxygen and carbon dioxide) can be provided from a supplemental source.

Where the bioreactor 12 is a polymer made from an additive manufacturing process, the sufficient access to gases can be via the polymer having sufficient gas permeability. The sufficient gas permeability will be understood by the skilled person in view of the disclosure herein. Gas permeability can be measured according to ASTM D1418 which generally utilizes a 25-mil sheet to measure gas permeability in cubic centimeters per cm² per second with one atmosphere (atm) pressure difference. In one or more embodiments, bioreactor 12 has a gas permeability relative to air of at least 0.2, in other embodiments at least 0.3, and in other embodiments at least 0.4. In one or more embodiments, bioreactor 12 has a gas permeability relative to air of from about 0.2 to about 0.5, in other embodiments from about 0.25 to about 0.4, and in other embodiments from about 0.3 to about 0.4. In one or more embodiments, bioreactor 12 has a gas permeability relative to oxygen of at least 0.3, in other embodiments at least 0.4, in other embodiments at least 0.5, and in other embodiments at least 0.6. In one or more embodiments, bioreactor 12 has a gas permeability relative to oxygen of from about 0.3 to about 0.8, in other embodiments from about 0.4 to about 0.7, and in other embodiments from about 0.5 to about 0.65. In one or more embodiments, bioreactor 12 has a gas permeability relative to carbon dioxide of at least 2, in other embodiments at least 2.5, in other embodiments at least 3, and in other embodiments at least 3.5. In one or more embodiments, bioreactor 12 has a gas permeability relative to carbon dioxide of from about 2 to about 4, in other embodiments from about 2.5 to about 4, and in other embodiments from about 2.5 to about 3.5. Stated again, units for gas permeability are cubic centimeters per cm² per second with one atmosphere (atm) pressure difference.

3D-printed structures can be used as the bioreactor 12, and the 3D-printed structure should be gas permeable, biocompatible, and mechanically flexible. Exemplary 3D-printing techniques, which can also be referred to as additive manufacturing, include extrusion-based techniques such as fused filament fabrication and direct ink writing (DIW). Further details of additive manufacturing will be generally known to the skilled person. A final size difference between a printed bioreactor 12 and a corresponding digital model can be less than 5%, or less than 3%, or less than 2%.

An exemplary material for bioreactor 12 is a silicone-based material. Silicone-based ink can be used for the additive manufacturing. Silicone materials generally have relatively high gas permeability compared with other elastomers for the aerobic biosynthesis of bacterial cellulose and for permeation of atmospheric carbon dioxide for subsequent carbonate biomineralization. Silicone materials also generally have a hydrophobic surface which can facilitate the separation of the biosynthesized composite 32 from the bioreactor 12. Silicone materials further generally have high biocompatibility, stability, and mechanical flexibility, which enables the creation of stable and complex 3D structures. The silicone material can be a mixture of two silicone elastomers. The silicone material can be a polydimethylsiloxane (PDMS) elastomer or a mixture of polydimethylsiloxane elastomers.

As mentioned above, bioreactor 12 includes shaped hollow interior 14 for loading the bacterial suspensions. Shaped hollow interior 14 can be a variety of shapes, such as a bone. The shaped hollow interior 14 should be inverse of the desired shape, so that after the microbial biosynthesis, the desired shape is generated. Where a bone shape is desired for a tissue engineering application, the exact geometry of shaped hollow interior 14 and bioreactor 12 can match an individual bone of a patient bone pattern as derived from a corresponding medical imaging technique, such as computed tomography (CT) scan or magnetic resonance imaging (MRI).

As mentioned above, a first culture 18 is added to bioreactor 12, which first culture 18 includes a first bacteria 20 capable of producing a structural polysaccharide such as bacterial cellulose. Aspects of the first culture 18, such as suitable nutrients, will be generally known to the skilled person in view of the present disclosure, and an exemplary composition for first culture 18 is a mannitol-based liquid medium, such as commonly known as MHS. The first bacteria 20 may include Gram-positive or Gram-negative bacteria. The first bacteria 20 may be vegetative or spores. The first bacteria 20 may include naturally occurring bacteria, modified bacteria, genetically modified bacteria, or combinations thereof. Any modification for first bacteria 20 may be for increased production of the structural polysaccharide. Exemplary bacteria for first bacteria 20 include K. xylinus, Acetobacter, Azotobacter, Rhizobium, Pseudomonas, Salmonella, Alcaligenes, Sarcina ventriculi, A. xylinum, A. hansenii, and A. pasteurianus.

The incubation conditions, which may also be referred to as culturing, for first culture 18 will be generally known to the skilled person in view of the disclosure herein. The incubation can include the bioreactor 12 providing control mechanisms, and/or being within a further controlled environment, such as a chamber, for control of the conditions, such as temperature and humidity. The incubation conditions can include utilizing a desired temperature, such as at least or about 25° C., at least or about 28° C., or at least or about 30° C. In these or other embodiments, the temperature can be from about 24° C. to about 32° C., or from about 25° C. to about 30° C., or from about 26° C. to about 28° C. The incubation conditions can include utilizing a certain humidity, such as at least or about 60%, at least or about 70%, at least or about 80%, or at least or about 90%. The incubation conditions can include a certain timeframe, such as at least or about 1 day, at least or about 3 days, at least or about 5 days, or at least or about 7 days.

During the incubation of first culture 18, first bacteria 20 will biologically produce a structural polysaccharide such as bacterial cellulose. The structural polysaccharide will be produced in the form of nanofibers in a network structure (i.e., organic network structure 22). The nanofibers can have a mean average diameter of about 50 nm and lengths exceeding hundreds of micrometers (e.g., greater than 200 mm, or greater than 300 mm, or greater than 400 mm). The properties of the nanofibers and corresponding organic network structure 22, such as thickness and porosity, can be tuned by adjusting the culturing time and oxygen content in bioreactor 12. With specific regard to bacterial cellulose (BC), bacterial cellulose has high mechanical strength, with up to 2 GPa tensile strength and 138 GPa Young's modulus. This strength is close to that of natural bone tissue and is higher than conventional biopolymers, such as collagen, silk, chitosan, and amyloid.

As mentioned above, following production of organic network structure 22, a second culture 24 is added to bioreactor 12 which includes a second bacteria 26 capable of mineralization of calcium carbonate (CaCO₃) 28. Aspects of the second culture 24, such as suitable nutrients, will be generally known to the skilled person in view of the present disclosure, and exemplary compositions for second culture 24 include those commonly known as Hestrin-Schramm medium (HS) and minimal B4 medium (MB4). The second bacteria 26 may include Gram-positive or Gram-negative bacteria. The second bacteria 26 may be vegetative or spores. The second bacteria 26 may include naturally occurring bacteria, modified bacteria, genetically modified bacteria, or combinations thereof. Any modification for second bacteria 26 may be for increased production of the calcium carbonate. Exemplary bacteria for second bacteria 26 include genera of Bacillus, Sporosarcina, Myxococcus, and Pseudomonas. Exemplary bacteria for second bacteria 26 include species of Bacillus simplex, such as B. simplex P6A, Bacillus pumillus, Bacillus megaterium, Sporosarcina pasteurii, Myxococcus xanthus, and Pseudomonas aeruginosa.

The incubation conditions, which may also be referred to as culturing, for second culture 24 will be generally known to the skilled person in view of the disclosure herein. The incubation can include the bioreactor 12 providing control mechanisms, and/or being within a further controlled environment, such as a chamber, for control of the conditions, such as temperature and humidity. The incubation conditions can include utilizing a desired temperature, such as at least or about 25° C., at least or about 28° C., or at least or about 30° C. In these or other embodiments, the temperature can be from about 24° C. to about 32° C., or from about 25° C. to about 30° C., or from about 26° C. to about 28° C. The incubation conditions can include utilizing a certain humidity, such as at least or about 60%, at least or about 70%, at least or about 80%, or at least or about 90%. The incubation conditions can include a certain timeframe, such as at least or about 1 day, at least or about 3 days, at least or about 5 days, or at least or about 7 days.

For the microbial synthesis of calcium carbonate by second bacteria 26, second bacteria 26 will exhibit mineralization of calcium carbonate (CaCO₃) via microbial-induced carbonate precipitation (MICP). Generally speaking, bacteria-induced mineralization will occur by second culture 24 and/or bioreactor 12 being adapted to provide metabolism-driven changes adapted to favor crystal nucleation and growth. Microbial-induced carbonate precipitation includes certain types of bacteria being capable of producing calcium carbonate. There are several mechanisms for microbial-induced carbonate precipitation, including the calcium toxicity mechanism, urea hydrolysis, ammonification of amino acids, denitrification, and photosynthesis. Aspects of these mechanisms and suitable bacteria will be generally known to the skilled person in view of the present disclosure. With reference to the above exemplary species for second bacteria 26, Bacillus simplex P6A utilizes the calcium toxicity mechanism; Bacillus pumillus, Bacillus megaterium, and Sporosarcina pasteurii utilize urea hydrolysis; Myxococcus xanthus utilizes ammonification of amino acids; and Pseudomonas aeruginosa utilizes denitrification. Further details are disclosed in Frankel, R. B., & Bazylinski, D. A. (2003); Biologically induced mineralization by bacteria; Reviews in mineralogy and geochemistry, 54(1), 95-114, which is incorporated herein by reference in this regard.

Where the calcium toxicity mechanism is relied on, a calcium source is provided in second culture 24, such that the calcium toxicity mechanism will result in the homeostatic maintenance of intracellular Ca²⁺ concentration by second bacteria 26 via the ChaA transporter protein. This process also relies on carbon dioxide via atmospheric or generated carbon dioxide. Bicarbonate ions are generated from the carbon dioxide using the YadF carbonic anhydrase, which derives the bicarbonate ions (HCO³⁻) from the carbon dioxide. As further description, the second bacteria 26 is stressed with Ca²⁺, which causes second bacteria 26 to precipitate calcium carbonate to remove excess Ca²⁺ ions. These carbonates preferentially precipitate (i.e., as particles 28) on polymeric surfaces, such as the nanofibers of organic network structure 22, resulting in the polymeric matrix of organic network structure 22 becoming decorated with particles 28. Further details regarding the calcium toxicity mechanism are disclosed in Banks, et al. 2010. Bacterial Calcium Carbonate Precipitation in Cave Environments: A Function of Calcium Homeostasis; Geomicrobiology Journal 27(5): 444-454 and U.S. Pat. No. 11,396,604, which are incorporated herein by reference in this regard. A calcium source for second culture 24 can be any suitable calcium containing material. Exemplary calcium sources may include an organic calcium salt, an inorganic calcium salt, or combinations thereof. Exemplary calcium salts include calcium acetate, calcium propionate, and calcium chloride.

Calcium carbonate has certain polymorphs, and the particular one or more polymorphs which occur as calcium carbonate particles 28 can depend on the media used for second culture 24. In one or more embodiments, calcium carbonate particles 28 are predominantly or entirely calcite. In one or more embodiments, calcium carbonate particles 28 are predominantly or entirely vaterite. In one or more embodiments, calcium carbonate particles 28 are a combination of calcite and vaterite. The media utilized for second culture 24 can affect calcium carbonate particles 28. For example, HS media may result in predominantly vaterite structure, and MB4 media may result in predominantly calcite structure. Also, MB4 media may result in a beads-on-a-string morphology, and HS media may result in a smoother calcium carbonate inorganic coating layer on the nanofibers. Further, the calcium carbonate particles generated from HS media will generally be larger in size and have rougher surface, where calcium carbonate particles generated from MB4 media will generally have more uniform size and well-defined crystalline morphology. This all allows for potential adaptation for different applications.

The method 10 also provides for controlling the density, porosity, and internal structure of the generated composites 32, such as by tuning the microbial growth conditions and number of rounds of cultivation. That is, the first culture 18 and second culture 24 steps can each be repeated. Multiple cycles of microbial biosynthesis in the bioreactor 12 will generally form 3D composites 32 with higher density than that from only one cycle. Though only one cycle may be preferred for certain applications. Where a bone-mimetic structure is desired for composite 32, additional cycles of microbial biosynthesis can lead to denser structure while still maintaining sufficient porosity in order to better mimic natural bone. In a similar manner, the weight ratio of BC to CaCO₃ can be further tuned by adjusting the duration of each bacteria culturing and the number of cycles. That is, the first culture 18 step might be longer than the second culture 24 step to increase the relative weight of the bacterial cellulose in the composite, or the second culture 24 step might be longer than the first culture 18 step to increase the relative weight of the inorganic component in the composite.

As mentioned above, the composite 32 may be utilized after the desired formation of calcium carbonate particles 28 for mineralized organic network structure 30 without modification of the calcium carbonate particles 28. These composites 32 without modification of the calcium carbonate particles 28 can be designed for desired properties, such as modulus, tensile strength, and elongation at break. The composition of composite 32, where unmodified, is tunable for a desired application. The composition of composite 32, where unmodified, can be adapted to generally match the composition of human bone. The composition of composite 32, where unmodified, can include about 50 to 80 wt. %, or about 60 to 75 wt. %, or about 65 to 75 wt. %, or about 75 to 85 wt. % of inorganic material. The composition of composite 32, where unmodified, can include about 20 to 50 wt. %, or about 20 to 40 wt. %, or about 30 to 40 wt. %, or about 30 to 35 wt. % of organic material.

The porosity of composite 32, where unmodified, can be adapted for a desired application, such as tissue engineering. The porosity of composite 32, where unmodified, can be characterized relative to density, where the density can be about 0.02 g/cm³, or about 0.06 g/cm³, or about 0.10 g/cm³. In these or other embodiments, the density of composite 32, where unmodified, can be from about 0.02 g/cm³ to about 0.15 g/cm³, or from about 0.04 g/cm³ to about 0.12 g/cm³, or from about 0.06 g/cm³ to about 0.10 g/cm³, or from about 0.02 g/cm³ to about 0.06 g/cm³.

For certain end applications for composite 32, composite 32 can be modified either before or after removal from bioreactor 12. These composites 32 with modification of the calcium carbonate particles 28 can be designed for desired properties, such as modulus, tensile strength, and elongation at break. The composition of composite 32, where modified, is tunable for a desired application. The composition of composite 32, where modified, can be adapted to generally match the composition of human bone. The composition of composite 32, where modified, can include about 50 to 80 wt. %, or about 60 to 75 wt. %, or about 65 to 75 wt. %, or about 75 to 85 wt. % of inorganic material. The composition of composite 32, where modified, can include about 20 to 50 wt. %, or about 20 to 40 wt. %, or about 30 to 40 wt. %, or about 30 to 35 wt. % of organic material.

The porosity of composite 32, where modified, can be adapted for a desired application, such as tissue engineering. The porosity of composite 32, where modified, can be characterized relative to density, where the density can be about 0.02 g/cm³, or about 0.06 g/cm³, or about 0.10 g/cm³. In these or other embodiments, the density of composite 32, where modified, can be from about 0.02 g/cm³ to about 0.15 g/cm³, or from about 0.04 g/cm³ to about 0.12 g/cm³, or from about 0.06 g/cm³ to about 0.10 g/cm³, or from about 0.02 g/cm³ to about 0.06 g/cm³.

An exemplary material for a modified inorganic component is calcium hydroxyapatite, which can provide improved biocompatibility. This chemical conversion from calcium carbonate to calcium hydroxyapatite has the advantages of being simple, occurring in mild conditions, and generating limited byproducts. To chemically convert the calcium carbonate particles to calcium hydroxyapatite particles, the composite 32 as an unmodified composite can be combined with ammonium phosphate, such as by immersion in ammonium phosphate solution. This can include certain reaction conditions, such as heating to about 60° C. under ambient pressure for about 4 hours. The ratio of components can generally follow the stoichiometric ratio based on the reaction: 10CaCO₃+6NH₄(H₂PO₄)+2H₂O→Ca₁₀(PO₄)₆(OH)₂+3(NH₄)2CO₃+7H₂CO₃. This will result in composite 32 as a modified composite having calcium hydroxyapatite particles.

As mentioned above, composite 32, which can be a modified composite 32, can be treated for removal of bacterial endotoxin. For potential tissue engineering applications, complete removal of bacterial endotoxin from the composite 32 can be critical. The main components that may be released after disintegration of bacteria include exopolysaccharides (EPS), peptidoglycan, and lipopolysaccharide (LPS). EPS from the bacterial debris are water-soluble, biodegradable, nontoxic, and biocompatible, and they can be conveniently removed with sodium hydroxide (NaOH) treatment and water washing. Peptidoglycan can be easily removed by lysozyme due to its ability to hydrolyze the β-1,4-glycosidic bond. The complex structure of LPS can make it more challenging to remove. For tissue engineering or implantation, the LPS level should be <0.5 EU/mL according to FDA standards for medical devices. An exemplary technique includes extended NaOH treatment of the composite 32 using a perfusion process for about 2 weeks followed by thorough water washing to effectively decrease the LPS level down to about 0.1 EU/mL or less.

While certain advantages of embodiments of the present invention are disclosed above, other specific advantages are disclosed here. Embodiments of the present invention provide customizable composite structures. Embodiments of the present invention can reduce the necessary processing of bacterial cellulose, which can be important due to its highly crystalline structure. Embodiments of the present invention provide precise control of the structure and physical properties of the composites. Embodiments of the present invention provide energy efficient, green, and sustainable methods.

In light of the foregoing, it should be appreciated that the present invention advances the art by providing improvements for microbial biosynthesis of composites. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Materials

Mannitol powder, citric acid monohydrate, and ammonium phosphate monobasic were purchased from VWR chemicals. Yeast extract, peptone, sodium phosphate dibasic anhydrous, calcium acetate, calcium carbonate, and acetic acid were purchased from Fisher Scientific. Agar was purchased from Genesee Scientific. Sodium hydroxide was obtained from Sigma-Aldrich. Trypticase soya broth (TSB) was obtained from Becton Dickinson and Company. Sylgard 184 elastomer and SE 1700 elastomer were obtained from Dow Chemicals. All chemicals were used as received without further purification.

### Bacteria

K. xylinus 700178 was purchased from the American Type Culture Collection (ATCC). The mannitol-based liquid medium (MHS) for K. xylinus contained mannitol 20 g/L, yeast extract 5 g/L, peptone 5 g/L, Na₂HPO₄2.7 g/L, and citric acid 1.5 g/L, with pH adjusted to 5.0 using acetic acid and sodium hydroxide. The culturing of K. xylinus was conducted statically at 28° C. and 80% humidity. The culturing time varied from 5 to 7 days.

B. simplex P6A was originally obtained from a cave environment. The minimal B4 (MB4) liquid medium for carbonate precipitation contained yeast extract 4 g/L and calcium acetate 2.5 g/L with pH adjusted to 7.2 using acetic acid and sodium hydroxide. The culturing of B. simplex P6A was conducted under shaking at 100 rpm. The culturing time varied from 4 to 7 days.

### 3D Printing

The direct ink writing (DIW) 3D printing ink contained a mixture of low-viscosity Sylgard 184 elastomer and nonflowing SE 1700 elastomer. The two materials were first mixed with their own curing agents in a 10:1 (w/w) ratio. Then, the SE 1700 elastomer/curing agent mixture and the Sylgard 184 elastomer/curing agent mixture were mixed at a weight ratio of 8:2 to obtain the printable ink. For the DIW 3D printing, the ink was loaded in a 3 mL syringe barrel and then centrifuged at 6,000 rpm for 10 minutes to remove the trapped air bubbles.

3D printing was conducted on a DIW 3D printer (Cellink 3D Bioprinter), which was connected to a compressor with pressure control. The 3D models were designed in Solidworks or Tinkercad, and the human bone model was obtained from the National Institutes of Health (NIH) 3D Print Exchange. The 3D printing path (G-code) was generated by slicing the STL files in Slic3r and then sent to the 3D printer through Repitier software. The nozzle inner diameter used was 600 μm (20G), the printing pressure was 130 to 140 kPa, and the speed was 180 mm/min. After DIW 3D printing, the structures were cured by heating in an oven at 60° C. for 24 h.

### BC-CaCO3 Composites

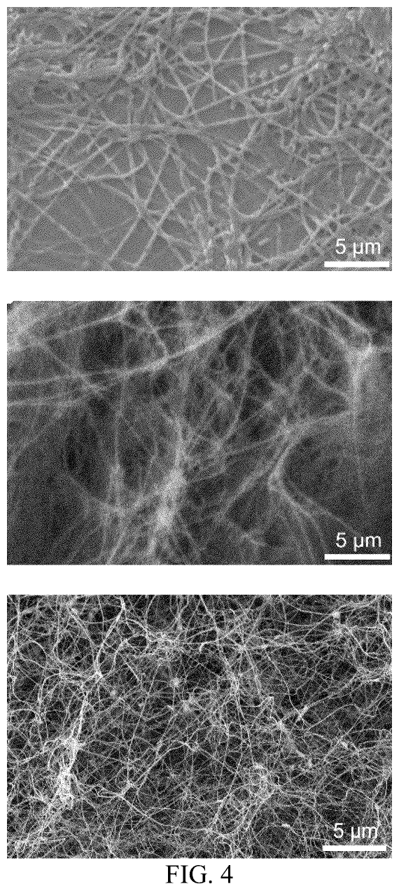

3D BC-CaCO₃ composite structures were fabricated by microbial biosynthesis in the 3D-printed silicone reactors. In the first step, an overnight culture of K. xylinus in MHS was used to establish a culture suspension of OD₆₀₀ 0.6 in fresh MHS. This culture was pipetted into a sterilized silicone reactor and incubated for 5 days at 28° C. and 80% humidity. This produced the bacterial cellulose network shown in FIG. 2. FIG. 4 also shows SEM images of bacterial cellulose films made with different culturing times, increasing from 1 day in the top image, 3 days in the middle image, to 5 days in the bottom image.

For the 5 day incubation material in the silicone reactor, the K. xylinus culture was removed, and the silicone reactor was rinsed with sterile water. A 100 μL inoculum of B. simplex P6A overnight culture in TSB media was combined with MB4 media within the silicone reactor. This second culture was incubated for 7 days at 28° C. and 80% humidity. These two steps represented one cycle of microbial biosynthesis.

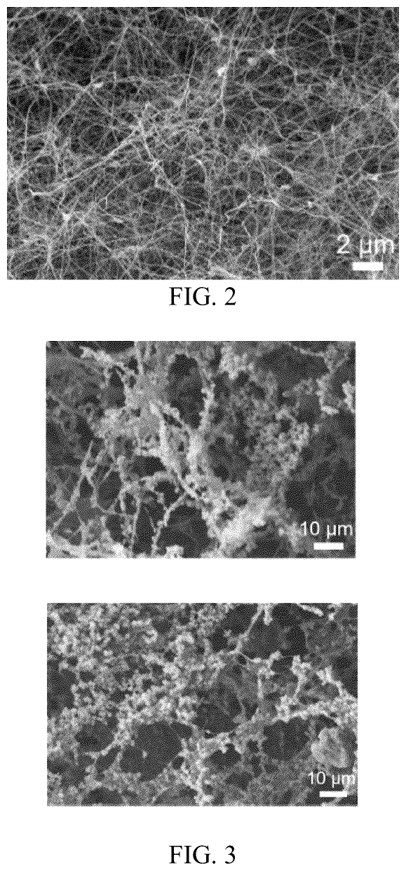

The microbial biosynthesis was repeated for 1.5 cycles (the last step is an additional K. xylinus step) or 2 cycles (repeating all steps twice), which could be an even higher number of cycles, to get more condensed composite structures. FIG. 3 shows SEM images of produced bacterial cellulose and calcium carbonate composites, with the top image showing a composite prepared by a one-cycle biosynthesis and the bottom image showing a composite prepared by two-cycle biosynthesis. After completion of the biosynthesis, the samples were freeze-dried at −50° C. for 24 hours to maintain the original size and shape. The freeze-dried samples were then separated from the silicone bioreactors for further study and characterization.

The composition and structure of the BC-CaCO₃ composites were investigated. Thermogravimetric analysis (TGA) results provided thermal stability and weight ratios of the composites. Pure BC has complete thermal degradation when heated to 450° C. and above, while the biosynthesized BC-CaCO₃ composites had substantial residual weight after 600° C. depending on the synthesis cycles, which corresponds to the inorganic (CaCO₃) component. The one cycle of microbial growth (K. xylinus, followed by B. simplex) showed a residual weight of 23% after heating to 600° C., while the 1.5-cycle composite (K. xylinus and B. simplex growth, followed by another round of K. xylinus growth) showed a residual weight of 20% at 600° C. This is likely due to one more cycle of BC synthesis decreasing the relative content of CaCO₃, which does not decompose until >850° C. The two-cycle composite showed a higher residual weight of CaCO₃ content of 33% at 600° C. The weight ratio of BC to CaCO₃ can be further tuned by adjusting the duration of each bacteria culturing and the number of cycles.

Fourier transform infrared spectroscopy (FTIR) was used to study the chemical composition and its changes after biomineralization. Pristine BC showed characteristic peaks for cellulose at 3350 cm⁻¹ (stretching of O—H bonds), 2919 cm⁻¹ (C—H stretching), 1427 cm⁻¹ (asymmetric angular deformation of C—H bonds), 1372 cm⁻¹ (symmetric angular deformation of C—H bonds), 1162 cm⁻¹ (asymmetrical stretching of C—O—C glycoside bonds), and 1058 and 1034 cm⁻¹ (C—O—C and C—O—H stretching vibration of polysaccharide). After the microbial biomineralization, the BC-CaCO₃ composites had all the peaks from BC and with two new peaks at 1420 and 871 cm⁻¹, which originate from calcium carbonate. X-ray diffraction (XRD) was used to characterize the crystalline structure of BC and the composites. Pristine BC showed two main peaks at 14.7 and 22.8°, which correspond to the (100) and (110) crystal planes of cellulose. For the BC-CaCO₃ composites, three new peaks at 25.3, 29.5, and 31.8° appeared, which correspond to the (110), (104), and (114) planes of CaCO₃. Those quantitative structural characterization and morphological studies showed successful fabrication of hierarchical BC-CaCO₃ composites that allow precisely controlled structure and geometry.

### BC-CaHA Composites

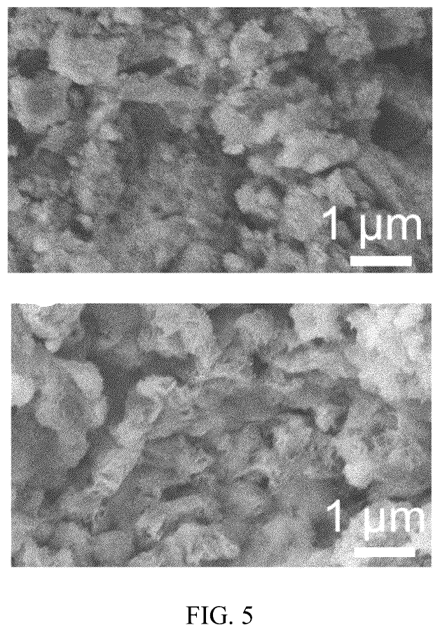

To convert the biosynthesized BC-CaCO₃ composites into biocompatible BC-CaHA composites with bone tissue engineering application, a convenient one-step method was used. The BC-CaCO₃ composite structure was immersed in an ammonium phosphate solution with a calculated stoichiometric ratio following the reaction: 10CaCO₃+6NH₄(H₂PO₄)+2H₂O→Ca₁₀(PO₄)₆(OH)₂+3(NH₄)2CO₃+7H₂CO₃. The reaction was conducted at 60° C. for 4 hours. Afterward, the sample was rinsed thoroughly with deionized (DI) water and further freeze-dried. This conversion is shown in FIG. 5.

The success of the chemical conversion of the composites was confirmed by FTIR and XRD analyses. After the conversion, the FTIR spectrum of BC-CaHA showed a strong peak at 1027 cm⁻¹, which originates from phosphate groups, and the original peaks for carbonate (871 and 1420 cm⁻¹) largely disappear. Moreover, after the conversion, the XRD peaks for CaCO₃ largely disappeared, and two new peaks at 26.0 and 32.1° appeared, which correspond to the (002) and (211) planes of the hydroxyapatite crystal. In addition, SEM showed that after conversion, the nanoparticles showed an increase in surface roughness and the overall shape and size remained consistent.

Energy-dispersive X-ray spectroscopy (EDX) was used to characterize the chemical composition change during conversion. The main difference was the substantial increase in the phosphorus peak, as well as the increase in O/C peak intensity ratio after the conversion, which confirmed the hydroxyapatite formation. The conversion under mild conditions enables the preservation of the original size and shape of the 3D composites. The internal morphological change after conversion was studied by cross-sectional SEM. The original BC-CaCO₃ composites had near-spherical CaCO₃ nanoparticles dispersed in the BC network. After the conversion, the CaHA nanoparticles showed a spiky or branched morphology with average size decreased to below 500 nm and were still well dispersed in the BC matrix. The overall porosity did not change substantially during this process, which is important for applications in tissue engineering scaffolds. The stability of the BC-CaHA composites in PBS buffer was improved compared with that of the BC-CaCO₃ composites, which can be explained by the internal structural change and higher stability of CaHA.

### Characterization

Scanning electron microscopy (SEM) was conducted with a JEOL-7401 FE-SEM operating at 5.0 kV with an 8 mm working distance. Fourier transform infrared spectroscopy (FTIR) was conducted with either transmission mode (for powder samples) or attenuated total reflection mode (for thin-film samples). The FTIR spectra were collected with an accumulation of 32 scans at a resolution of 4 cm⁻¹ in the range of 4000 to 650 cm⁻¹. X-ray diffraction (XRD) was conducted with an Ultima IV X-ray diffractometer (Rigaku, Tokyo, Japan) operated at 40 kV and 35 mA, with a Cu Kα energy frequency (wavelength of 1.54 Å). Thermogravimetric analysis (TGA) was conducted with a TA Discovery 550; the temperature range was 20 to 700° C. with a heating rate of 20° C./min under a nitrogen atmosphere. Dynamic mechanical analysis (DMA) was conducted with a TA Q800 instrument. The DMA tensile measurements were conducted using rectangular thin films (20×5 mm) at a rate of 0.1 N/min. DMA compression tests were conducted with an RSA-G2 instrument with a constant linear rate of 0.1 mm/s. The optical density was measured by a HACH DR 2800 spectrophotometer.

The mechanical properties of the microbial-biosynthesized composites were studied by tensile testing. The representative stress-strain curves for the samples were obtained. Pristine BC had a modulus of 3.11 GPa, a tensile strength of 53 MPa, and an elongation at break of 2.0%. The biomineralized BC-CaCO₃ composite showed a decreased modulus of 1.15 GPa, a tensile strength of 25 MPa, and an elongation at break of 2.8%. The probable reason for the decrease in mechanical strength and modulus is the relatively large size of the CaCO₃ particles and their limited affinity for BC nanofibers. After chemical conversion to BC-HA, the composite showed a substantially improved modulus of 6.30 GPa, a tensile strength of 90 MPa, and an elongation at break of 1.9%. Those mechanical properties are close to human bones and can be further optimized or adjusted by tuning the internal structure and composition during microbial biosynthesis. Those tensile testing samples were in a dry state and prepared by compressing the as-synthesized composite membrane into a dense film. Compressive mechanical testing was also conducted on porous BC-CaCO₃ composites prepared by freeze-drying, and the results showed that both the porosity (or density) and the composition of the composites substantially affect their mechanical properties. Mechanical properties of the as-fabricated composites in wet hydrogel state were also studied.

### Shapes

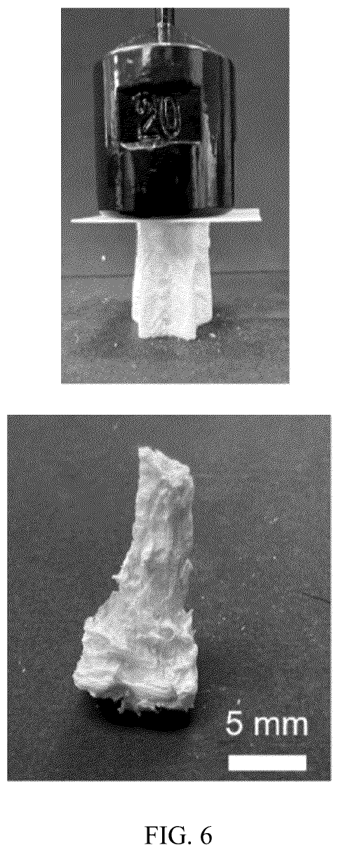

Composites with different shapes were created by using silicone bioreactors with different geometries. As shown in FIG. 6, a star cylinder-shaped BC-CaCO₃ composite was used to demonstrate the mechanical strength by placing a standard weight on top. The star cylinder-shaped BC-CaCO₃ composite withstood more than 2500 times its own weight (8 mg structure vs 20 g standard weight). A bone-shaped BC-CaCO₃ composite was also fabricated (bottom image of FIG. 6) and showed high shape fidelity compared to the digital model.

### Different Compositions

Additional mechanical properties were studied (compressive testing) for composites prepared by physical blending of BC and CaCO₃ with the composition and density match with those from microbial biosynthesis. Three types of BC-CaCO₃ composites were prepared and freeze-dried. The first type had 33 wt. % CaCO₃ and high porosity (with a density of 0.023 g/cm³), which matched with the 2-cycle biosynthesized sample; the second type had 33 wt. % CaCO₃ and a lower porosity (higher density of 0.10 g/cm³). To study the effect of ratio between BC to CaCO₃ on the composite mechanical properties, we also prepared a third type, which had 20 wt. % CaCO₃ and similar porosity with type II (with a density of 0.06 g/cm³). Compression tests were conducted on those three types of samples and the compressive stress-strain curves were obtained. From the results, the porosity (or density) had an effect on the composite mechanical properties. For the BC-CaCO₃ (33 wt. %) composite with high porosity, the compressive strength was 69.7 kPa, and compressive modulus was 49.2 kPa. With the decrease in porosity (or increase in density), the compressive strength increased to 93.1 kPa, and compressive modulus increased to 300.0 kPa. When comparing the two BC-CaCO3 composite with different CaCO₃ weight fraction (33 wt. % vs 20 wt. %), the one with lower CaCO3 content (20 wt. %) had a comparable compressive strength (100 kPa), but lower compressive modulus (92.0 kPa). It is noted that the BC-CaCO3 composites fabricated had substantially lower density or higher porosity than natural bones, which explains the relatively lower mechanical strength. The BC-CaHA composite structures are proposed to be used as tissue engineering scaffolds for osteoblasts, and such tissue engineered bones are expected to have substantially improved mechanical properties.

Various modifications and alterations that do not depart from the scope and spirit of this invention will become apparent to those skilled in the art. This invention is not to be duly limited to the illustrative embodiments set forth herein.

What is claimed is:

1. A method of microbial biosynthesis of a composite, the method comprising subjecting a first culture within a bioreactor to incubation conditions, the first culture including a first bacteria that exhibit production of a structural polysaccharide, the incubation conditions thereby causing the first bacteria to produce an organic network structure made of a produced structural polysaccharide;
adding a second culture to the bioreactor after the organic network structure is produced, the second culture including a second bacteria that exhibit mineralization of calcium carbonate (CaCO₃) via microbial-induced carbonate precipitation (MICP); and
allowing the second bacteria to produce calcium carbonate particles which precipitate on the organic network structure to produce a mineralized organic network structure as the composite.

2. The method of claim 1, where the bioreactor includes a hollow interior portion having an inverse shape of a desired shape of the composite.

3. The method of claim 2, where the first culture and the second culture completely fill the hollow interior portion such that the composite matches the desired shape.

4. The method of claim 3, where the desired shape is a bone shape.

5. The method of claim 4, where the bone shape is based on one or more of a computed tomography scan image and a magnetic resonance imaging image.

6. The method of claim 1, where the second bacteria exhibit mineralization of calcium carbonate via a calcium toxicity mechanism in the presence of a calcium source, the second culture further including the calcium source to achieve the calcium toxicity mechanism.

7. The method of claim 1, where the structural polysaccharide is bacterial cellulose.

8. The method of claim 1, where the organic network structure includes nanofibers with an average diameter of about 50 nanometers and lengths greater than 200 micrometers.

9. The method of claim 1, further comprising adding an additional amount of the first culture to the bioreactor.

10. The method of claim 1, further comprising adding an additional amount of the second culture to the bioreactor.

11. The method of claim 1, further comprising steps of removing a remainder of the first culture from the bioreactor, and rinsing the bioreactor with sterile water following the removing the remainder of the first culture.

12. The method of claim 1, further comprising a step of making the bioreactor by additive manufacturing.

13. The method of claim 12, where the additive manufacturing includes direct ink writing (DIW) a silicone-based ink.

14. The method of claim 1, where the bioreactor is made of a silicone-based material.

15. The method of claim 14, where the silicone-based material includes a mixture of polydimethylsiloxane elastomers.

16. The method of claim 1, where the bioreactor is a metal vessel.

17. The method of claim 1, further comprising freeze drying the mineralized organic network structure.

18. The method of claim 17, further comprising sanitizing the mineralized organic network structure after the freeze drying.

19. The method of claim 1, further comprising combining the mineralized organic network structure with ammonium phosphate to thereby convert the calcium carbonate particles to calcium hydroxyapatite particles to form a modified mineralized organic network structure as the composite.

20. The method of claim 19, further comprising freeze drying the modified mineralized organic network structure and thereafter sanitizing the modified mineralized organic network structure.
